(19) United States
(12) Patent Application Publication
(10) Pub. No.: US 2023/0404535 A1
(43) Pub. Date: Dec. 21, 2023

(54) ULTRASONIC DIAGNOSTIC APPARATUS, METHOD FOR CONTROLLING ULTRASONIC DIAGNOSTIC APPARATUS, AND CONTROL PROGRAM FOR ULTRASONIC DIAGNOSTIC APPARATUS

(71) Applicant: Konica Minolta, Inc., Tokyo (JP)

(72) Inventors: AKIHIRO KAWABATA, Tokyo (JP); SHUHEI OKUDA, Tokyo (JP)

(21) Appl. No.: 18/205,061

(22) Filed: Jun. 2, 2023

(30) Foreign Application Priority Data
Jun. 17, 2022 (JP) 2022-098290

(51) Int. Cl. A61B 8/08; A61B 8/06; A61B 8/14

(52) U.S. Cl. CPC A61B 8/488; A61B 8/06; A61B 8/14; A61B 8/5207

(57) ABSTRACT

An ultrasonic diagnostic apparatus includes: a transceiver that transmits and receives an ultrasonic wave to obtain a reception signal related to an ultrasonic echo; a detector that detects the reception signal and generates a Doppler signal; a filtering part that performs filter processing of cutting or suppressing a low-frequency component included in the Doppler signal and generates a first filtered Doppler signal and a second filtered Doppler signal from the Doppler signal; an analyzer that performs frequency analysis on the first filtered Doppler signal to generate an image signal; and a generator that generates a sound signal based on the second filtered Doppler signal.

The entire disclosure of Japanese patent Application No. 2022-098290, filed on Jun. 17, 2022, is incorporated herein by reference in its entirety.

### BACKGROUND

### Technological Field

The present disclosure relates to an ultrasonic diagnostic apparatus, a method for controlling an ultrasonic diagnostic apparatus, and a control program for an ultrasonic diagnostic apparatus.

### Description of the Related Art

Conventionally, there is known an ultrasonic diagnostic apparatus having a Doppler mode which is an image mode for measuring a blood flow velocity or the like of a subject using the principle of Doppler and spectrally displaying a change in the velocity of the blood flow on the basis of a Doppler waveform obtained by the measurement. In such an ultrasonic diagnostic apparatus, examples of the Doppler mode include a pulse wave Doppler (PWD) mode, a continuous wave Doppler (CWD) mode, and a tissue Doppler (TDI) mode. For example, in the PWD mode, the ultrasonic pulse is transmitted into the living body, and the Doppler information extracted from the observation area (Doppler sample gate) for measuring the Doppler signal set to a specific depth is subjected to the frequency analysis, and the Doppler waveform is formed from the Doppler spectrum (representing the signal intensity for each blood flow velocity) obtained by the frequency analysis. Further, for example, in the CWD mode, a continuous wave ultrasonic wave is transmitted, and a reflected wave from the ultrasonic beam axis is received. Doppler information is extracted from the reception signal obtained as a result, and a Doppler waveform is formed in the same manner as described above.

FIG. 1A is a diagram illustrating an example of a Doppler waveform.

The Doppler waveform is information on a temporal change in the velocity (that is, the Doppler shift frequency) of the motion of the observation target generated on the basis of the time-series Doppler spectrum. For example, as illustrated in FIG. 1A, the Doppler waveform is expressed by a waveform in which time is on the horizontal axis, velocity (that is, the Doppler shift frequency) is on the vertical axis, and signal intensity (also referred to as power) of each velocity (that is, the frequency component) is luminance (gradation).

In FIG. 1A, a circled region represents a Doppler sample gate, and a Doppler waveform is formed by capturing a blood flow signal in the region. Note that, in FIG. 1A, a straight line on the illustrated time axis is referred to as a baseline, the velocity on the upper side in the drawing with respect to the baseline represents the velocity of blood flow toward the probe, and the velocity on the lower side in the drawing with respect to the baseline represents the velocity of blood flow away from the probe.

In the Doppler signal, there is an unnecessary signal (referred to as clutter, clutter signal, or clutter component) from a slow-moving object such as a wall of the heart. Clutter, which is a strong, low-frequency, unwanted signal, is removed by a wall motion filter, usually comprised of a high-pass filter, because clutter interferes with detecting weak blood flow signals. Therefore, the wall motion filter may be regarded as a filter for extracting the blood flow component by cutting the tissue component of the Doppler signal. Note that the wall motion filter may be referred to as a wall filter, a clutter filter, a moving target indicator (MTI) filter, or the like.

For example, JP 2-264644 A discloses a technique for removing clutter.

In the technique disclosed in JP 2-264644 A, in order to remove clutter, filtering (filter processing) is performed by applying a common band-pass filter (corresponding to a wall motion filter) to an image and sound, and a Doppler signal from which clutter has been removed is subjected to frequency analysis by the band-pass filter to generate a spectrum signal. Further, in order to remove the clutter of the low frequency from the Doppler sound, the clutter is filtered in the spectrum signal, and the filtered signal is subjected to inverse Fourier transform to generate the Doppler sound. In this technique, since the Doppler sound is generated from the spectrum signal in this manner, processing of synthesizing the inverse Fourier transform result with the Doppler sound occurs, and the processing becomes complicated.

### SUMMARY

The present disclosure has been made in view of the above problems, and an object of the present disclosure is to provide an ultrasonic diagnostic apparatus, a method for controlling an ultrasonic diagnostic apparatus, and a control program for an ultrasonic diagnostic apparatus capable of generating a Doppler sound from which a low-frequency clutter has been removed without complicating processing.

To achieve the abovementioned object, according to an aspect of the present invention, an ultrasonic diagnostic apparatus reflecting one aspect of the present invention comprises: a transceiver that transmits and receives an ultrasonic wave to obtain a reception signal related to an ultrasonic echo; a detector that detects the reception signal and generates a Doppler signal; a filtering part that performs filter processing of cutting or suppressing a low-frequency component included in the Doppler signal and generates a first filtered Doppler signal and a second filtered Doppler signal from the Doppler signal; an analyzer that performs frequency analysis on the first filtered Doppler signal to generate an image signal; and a generator that generates a sound signal based on the second filtered Doppler signal.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1A is a diagram illustrating an example of a Doppler waveform;

FIG. 1B is a diagram illustrating an example of a Doppler waveform;

FIG. 1C is a diagram illustrating an example of a Doppler waveform;

FIG. 2 is a diagram illustrating an example of an appearance of an ultrasonic diagnostic apparatus according to a first embodiment of the present disclosure;

FIG. 3 is a diagram illustrating an example of an overall configuration of the ultrasonic diagnostic apparatus according to the first embodiment of the present disclosure;

FIG. 4 is a diagram illustrating an example of a configuration of a Doppler signal processor of the ultrasonic diagnostic apparatus according to the first embodiment of the present disclosure;

FIG. 5A is a diagram illustrating an example of filter characteristics of a wall motion filter for image and a wall motion filter for sound according to the first embodiment of the present disclosure;

FIG. 5B is a diagram illustrating an example of filter characteristics of a wall motion filter for image and a wall motion filter for sound according to the first embodiment of the present disclosure;

FIG. 6 is a diagram illustrating an example of a cutoff frequency selection user interface according to the first embodiment of the present disclosure;

FIG. 7 is a flowchart illustrating an example of the operation of the ultrasonic diagnostic apparatus A according to the first embodiment of the present disclosure;

FIG. 8 is a diagram illustrating an example of a configuration of a Doppler signal processor of an ultrasonic diagnostic apparatus according to a second embodiment of the present disclosure;

FIG. 9A is a diagram illustrating an example of filter characteristics of a wall motion filter for shared and a wall motion filter for sound and filter characteristics obtained by synthesizing these filters according to the second embodiment of the present disclosure;

FIG. 9B is a diagram illustrating an example of filter characteristics of a wall motion filter for shared and a wall motion filter for sound and filter characteristics obtained by synthesizing these filters according to the second embodiment of the present disclosure;

FIG. 9C is a diagram illustrating an example of filter characteristics of a wall motion filter for shared and a wall motion filter for sound and filter characteristics obtained by synthesizing these filters according to the second embodiment of the present disclosure;

FIG. 10 is a diagram illustrating an example of a cutoff frequency selection user interface according to the second embodiment of the present disclosure; and

FIG. 11 is a flowchart illustrating an example of the operation of the ultrasonic diagnostic apparatus A according to the second embodiment of the present disclosure.

### DETAILED DESCRIPTION OF EMBODIMENTS

Hereinafter, one or more embodiments of the present invention will be described with reference to the drawings. However, the scope of the invention is not limited to the disclosed embodiments. Note that, in the present specification and the drawings, components having substantially the same function are denoted by the same reference numerals, and redundant description is omitted.

### First Embodiment

[Configuration of Ultrasonic Diagnostic Apparatus]

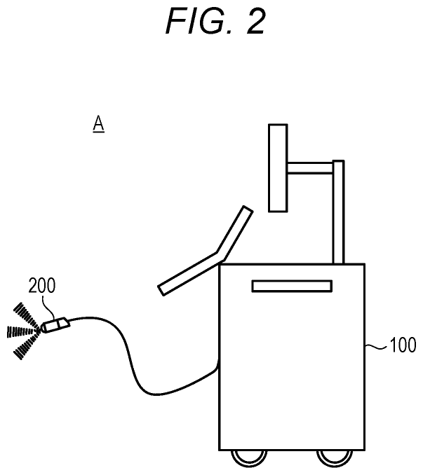

Hereinafter, a configuration of an ultrasonic diagnostic apparatus (hereinafter, referred to as an “ultrasonic diagnostic apparatus A”) according to a first embodiment of the present disclosure will be described with reference to FIGS. 2 to 4.

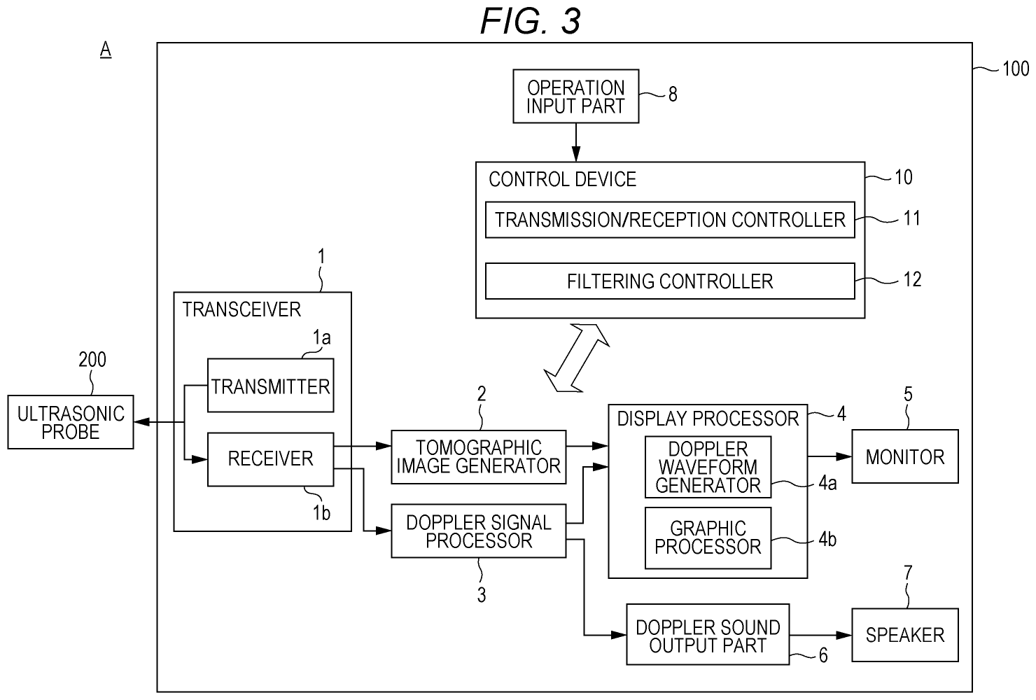

FIG. 2 is a diagram illustrating an example of an appearance of the ultrasonic diagnostic apparatus A. FIG. 3 is a diagram illustrating an example of an overall configuration of the ultrasonic diagnostic apparatus A.

The ultrasonic diagnostic apparatus A is used to visualize the shape, property, or dynamics in the subject as an ultrasonic image and perform image diagnosis. Note that, in the present embodiment, a blood flow flowing in a blood vessel of a subject is taken as an example of an observation target of the ultrasonic diagnostic apparatus A. However, the observation target of the ultrasonic diagnostic apparatus A may be a tissue other than a blood flow of a subject.

As illustrated in FIG. 2, the ultrasonic diagnostic apparatus A includes an ultrasonic diagnostic apparatus main body 100 and an ultrasonic probe 200.

The ultrasonic probe 200 functions as an acoustic sensor that transmits an ultrasonic beam (here, about 1 to 30 MHz) to the inside of the subject (for example, human body), receives an ultrasonic echo reflected in the subject out of the transmitted ultrasonic beam, and converts the ultrasonic echo into an electric signal.

The user brings the transmission/reception surface of the ultrasonic beam of the ultrasonic probe 200 into contact with the subject and operates the ultrasonic diagnostic apparatus A to perform ultrasonic diagnosis. Note that any probe such as a convex probe, a linear probe, a sector probe, or a three-dimensional probe can be applied to the ultrasonic probe 200.

The ultrasonic probe 200 includes, for example, a plurality of transducers (for example, a piezoelectric element) arranged in a matrix, and a channel switching part (for example, a multiplexer) for controlling switching between on and off of drive states of the plurality of transducers individually or in units of blocks (hereinafter, referred to as “channels”). Each transducer of the ultrasonic probe 200 converts a voltage pulse generated in the ultrasonic diagnostic apparatus main body 100 (transmitter 1a) into an ultrasonic beam and transmits the ultrasonic beam into the subject, receives an ultrasonic echo reflected in the subject, converts the ultrasonic echo into an electric signal (hereinafter, referred to as a “reception signal”), and outputs the electric signal to the ultrasonic diagnostic apparatus main body 100 (receiver 1b).

The ultrasonic diagnostic apparatus main body 100 includes a transceiver 1 (transmitter 1a and receiver 1b), a tomographic image generator 2, a Doppler signal processor 3, a display processor 4, a monitor 5, a Doppler sound output part 6, a speaker 7, an operation input part 8, and a control device 10.

The transmitter 1a of the transceiver 1 (transceiver according to the present disclosure) is a transmission device that transmits a voltage pulse as a drive signal to the ultrasonic probe 200. The transmitter 1a includes, for example, a high-frequency pulse oscillator, a pulse setting part, and the like (none of them are illustrated). The transmitter 1a adjusts the voltage pulse generated by the high-frequency pulse oscillator to the voltage amplitude, the pulse width, and the transmission timing set by the pulse setting part, and transmits the voltage pulse for each channel of the ultrasonic probe 200.

The transmitter 1a includes a pulse setting part in each of the plurality of channels of the ultrasonic probe 200, and can set the voltage amplitude, the pulse width, and the transmission timing of the voltage pulse for each of the plurality of channels. For example, the transmitter 1a changes a target depth or generates a different pulse waveform by setting an appropriate delay time for a plurality of channels (for example, one wave pulse is transmitted in the B mode, and four wave pulses are transmitted in the PWD mode).

The receiver 1b of the transceiver 1 is a reception device that performs reception processing of a reception signal related to an ultrasonic echo generated by the ultrasonic probe 200. The receiver 1b includes a preamplifier, an AD converter, a reception beamformer, and a processing system switching part (all not illustrated).

The receiver 1b amplifies a reception signal related to a weak ultrasonic echo for each channel by a preamplifier, and converts the reception signal into a digital signal by an AD converter. Then, the receiver 1b combines the reception signals of the plurality of channels into one by phasing addition of the reception signals of the respective channels by the reception beamformer to obtain acoustic beam data. In addition, the receiver 1b causes the processing system switching part to perform switching control of a destination to which the reception signal generated by the reception beamformer is transmitted, and outputs the reception signal to one of the tomographic image generator 2 and the Doppler signal processor 3 according to the operation mode to be executed.

The tomographic image generator 2 acquires the reception signal from the receiver 1b during the B-mode operation, and generates a tomographic image (also referred to as a B-mode image) inside the subject.

For example, when the ultrasonic probe 200 transmits a pulsed ultrasonic beam in the depth direction, the tomographic image generator 2 temporally continuously accumulates signal intensity of an ultrasonic echo detected thereafter in the line memory. Then, as the ultrasonic beam from the ultrasonic probe 200 scans the inside of the subject, the tomographic image generator 2 sequentially accumulates the signal intensity of the ultrasonic echo at each scanning position in the line memory, and generates two-dimensional data in units of frames. Then, the tomographic image generator 2 generates a tomographic image by converting signal intensity of an ultrasonic echo detected at each position inside the subject into a luminance value.

The tomographic image generator 2 includes, for example, an envelope detection circuit, a dynamic filter, and a logarithmic compression circuit. The envelope detection circuit performs envelope detection on the reception signal to detect signal intensity. The logarithmic compression circuit performs logarithmic compression on the signal intensity of the reception signal detected by the envelope detection circuit. The dynamic filter is a band-pass filter in which a frequency characteristic is changed according to a depth, and removes a noise component included in a reception signal.

The Doppler signal processor 3 acquires the reception signal from the receiver 1b in the PWD mode, the CWD mode, and the TDI mode, and detects the Doppler shift frequency with respect to the transmission frequency of the ultrasonic echo from the blood flow. Then, the Doppler signal processor 3 sequentially outputs information related to the Doppler spectrum representing the signal intensity for each Doppler shift frequency (that is, for each velocity of the blood flow) to the display processor 4. Note that the velocity of the blood flow and the Doppler shift frequency are in a direct proportional relationship as in the following equation (1).

V=c/2 cos θ×Fd/F0 (1)

(where V is the blood flow velocity, F0 is the transmission frequency (or reception frequency) of the ultrasonic beam, Fd is the Doppler shift frequency, c is the in vivo sound velocity, and θ is the intersection angle between the beam direction of the ultrasonic beam and the blood flow direction)

For example, in the PWD mode operation, when the ultrasonic probe 200 transmits a pulsed ultrasonic beam at regular intervals according to a pulse repetition frequency, the Doppler signal processor 3 samples a reception signal related to an ultrasonic echo in synchronization with the pulse repetition frequency. Then, the Doppler signal processor 3 detects the Doppler shift frequency on the basis of, for example, the phase difference between the ultrasonic echo related to the n-th ultrasonic beam and the ultrasonic echo related to the (n+1)-th ultrasonic beam from the same sample gate position.

In addition, the Doppler signal processor 3 separates the directions (blood flow toward or away from ultrasonic probe 200) of the reception signal from the receiver 1b with respect to the ultrasonic probe 200. The Doppler signal processor 3 converts the frequency component of the separated reception signal into a frequency of an easily audible sound pitch as necessary. Then, the Doppler signal processor 3 executes post-processing such as DA conversion on the separated reception signal subjected to frequency conversion as necessary to generate a Doppler sound signal, and outputs the generated Doppler sound signal to the Doppler sound output part 6.

The Doppler signal processor 3 applies the wall motion filter for image to the Doppler signal before generating the Doppler spectrum, and applies the wall motion filter for sound to the Doppler signal before generating the Doppler sound signal. As described above, in the present embodiment, by separately applying the wall motion filter for image and the wall motion filter for sound to the Doppler signal, the Doppler sound from which the low-frequency clutter has been removed is generated while avoiding the complexity of the processing of generating the Doppler sound by performing the inverse Fourier transform on the filtered signal.

The display processor 4 acquires the tomographic image output from the tomographic image generator 2 and the Doppler spectrum output from the Doppler signal processor 3, and generates a display image to be displayed on the monitor 5.

The display processor 4 includes a Doppler waveform generator 4a and a graphic processor 4b.

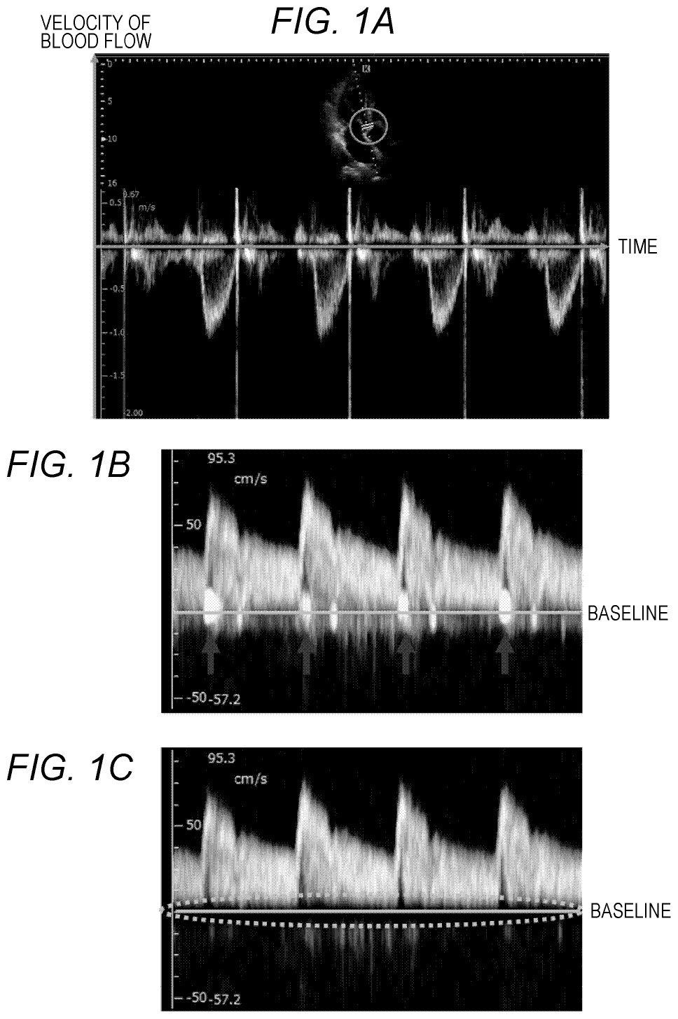

For example, the Doppler waveform generator 4a generates a Doppler waveform on the basis of a time-series Doppler spectrum sequentially output from the Doppler signal processor 3. As illustrated in FIG. 1A, the Doppler waveform is information of a temporal change in the velocity (that is, the Doppler shift frequency) of the movement of the observation target generated on the basis of the time-series Doppler spectrum. For example, the blood flow velocity at each time point is expressed in a form of a single line, and the signal intensity for each blood flow velocity (that is, for each Doppler shift frequency) is expressed by the magnitude of the luminance of the pixel.

The graphic processor 4b performs various types of image processing on the tomographic image output from the tomographic image generator 2 and the Doppler waveform image generated by the Doppler waveform generator 4a. Then, the graphic processor 4b generates a display image using the tomographic image and the Doppler waveform image.

The monitor 5 is a display that displays a display image generated by the display processor 4, a setting user interface (screen) that enables the user to set filter characteristics (for example, cutoff frequency or the like), and the like. The monitor 5 may be configured by, for example, a liquid crystal display.

The Doppler sound output part 6 generates a Doppler sound to be output from the speaker 7 by power-amplifying the Doppler sound signal output from the Doppler signal processor 3 with a power amplifier.

The speaker 7 is, for example, a speaker that outputs the Doppler sound from the Doppler sound output part 6 to the outside.

Note that the tomographic image generator 2, the Doppler signal processor 3, the display processor 4, and/or the Doppler sound output part 6 are realized by, for example, a digital arithmetic circuit including a digital signal processor (DSP) or the like. However, these configurations can be variously modified, and for example, some or all of them may be realized by a hardware circuit or may be realized by arithmetic processing according to a program.

The operation input part 8 is a user interface for the user to perform an input operation, and includes, for example, a push button switch, a keyboard, a mouse, and the like. The operation input part 8 converts an input operation performed by the user into an operation signal and inputs the operation signal to the control device 10.

The control device 10 exchanges signals with the ultrasonic probe 200, the transceiver 1, the tomographic image generator 2, the Doppler signal processor 3, the display processor 4, the monitor 5, the Doppler sound output part 6, the speaker 7, and the operation input part 8, and integrally controls them. Note that the control device 10 includes, for example, a central processing unit (CPU), a read only memory (ROM), a random access memory (RAM), and the like. Each function of the control device 10 is realized by the CPU referring to a control program or various data stored in the ROM or the RAM. However, it is a matter of course that some or all of the functions of the control device 10 can be realized not only by software processing but also by a dedicated hardware circuit or a combination thereof.

The control device 10 includes a transmission/reception controller 11 and a filtering controller 12.

The transmission/reception controller 11 determines the transmission/reception condition of the ultrasonic beam on the basis of the type (for example, a convex type, a sector type, a linear type, or the like) of the connected ultrasonic probe 200, the depth of the imaging target in the subject set by the user via the operation input part 8, the imaging mode (for example, the B mode, the PWD mode, the color Doppler mode, or the power Doppler mode), and the like. Then, the transmission/reception controller 11 controls each of the transmitter 1a and the receiver 1b so as to satisfy the transmission/reception conditions determined in this manner, and causes the ultrasonic probe 200 to transmit and receive ultrasonic waves.

The filtering controller 12 controls the wall motion filter and its filter processing on the basis of the filter characteristics of the wall motion filter (for example, a wall motion filter for image 3fi to be described later, or the like). Note that the filter characteristics of the wall motion filter may be set and applied by the user via the operation input part 8, or may be applied on the basis of the internal parameters of the ultrasonic diagnostic apparatus A.

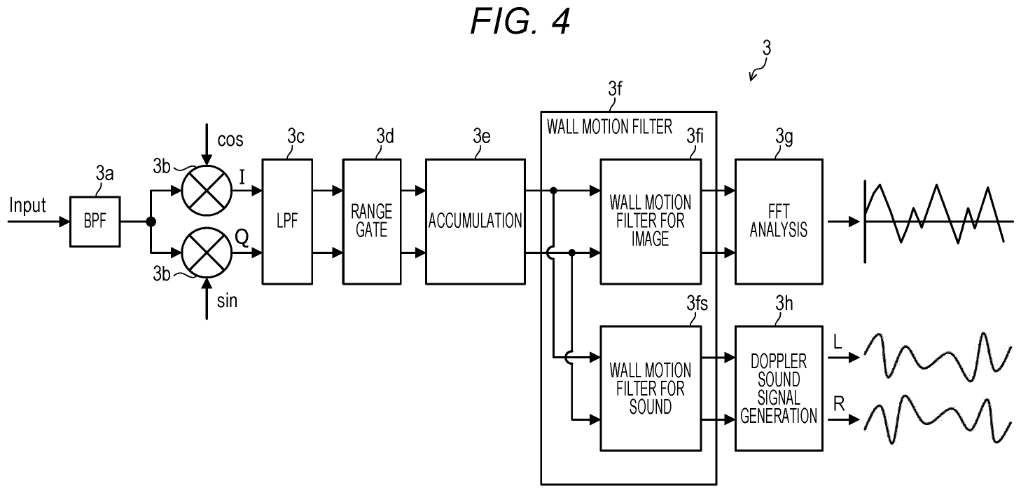

FIG. 4 is a diagram illustrating an example of a configuration of the Doppler signal processor 3 that executes PWD according to the first embodiment. The Doppler signal processor 3 that executes PWD includes, for example, a band-pass filter 3a, an orthogonal detector 3b, a low-pass filter 3c, a range gate 3d, an accumulation circuit 3e, a wall motion filter 3f, an FFT analyzer 3g, and a Doppler sound signal generator 3h.

The band-pass filter 3a removes unnecessary frequency components from the reception signal, and outputs the reception signal from which the unnecessary frequency components have been removed to the orthogonal detector 3b.

The orthogonal detector 3b mixes a reference signal having the same phase as the transmitted ultrasonic pulse and a reference signal having a phase different from that of the transmitted ultrasonic pulse by n/2 with respect to the reception signal output from the band-pass filter 3a, generates an orthogonal detection signal, and outputs the orthogonal detection signal to the low-pass filter 3c. The orthogonal detector 3b is an example of a detector according to the present disclosure. The orthogonal detection signal is an example of a Doppler signal or a Doppler shift signal according to the present disclosure.

The low-pass filter 3c removes a high-frequency component of the orthogonal detection signal output from the orthogonal detector 3b, generates a reception signal related to the Doppler shift frequency, and outputs the reception signal to the range gate 3d.

The range gate 3d acquires only an ultrasonic echo from the Doppler sample gate depth out of the reception signal output from the low-pass filter 3c, and outputs the ultrasonic echo to the accumulation circuit 3e.

The accumulation circuit 3e accumulates the reception signal output from the range gate 3d and outputs the accumulated signal to the wall motion filter 3f (wall motion filter for image 3fi and wall motion filter for sound 3fs to be described later).

The wall motion filter 3f performs filter processing of applying the wall motion filter for image 3fi and the wall motion filter for sound 3fs to cut or suppress a low-frequency component (clutter component) included in the reception signal output from the accumulation circuit 3e. The wall motion filter 3f is an example of a filtering part according to the present disclosure.

The wall motion filter 3f includes the wall motion filter for image 3fi and the wall motion filter for sound 3fs.

The wall motion filter for image 3fi may be configured as, for example, a high-pass filter. Under the control of the filtering controller 12, the wall motion filter for image 3fi performs filter processing for cutting or suppressing a low-frequency component (clutter component) included in the reception signal output from the accumulation circuit 3e on the basis of filter characteristics (for example, cutoff frequency or the like) applied for images. Then, the wall motion filter for image 3fi generates a reception signal in which the clutter component is cut or suppressed, and outputs the reception signal to the FFT analyzer 3g. The reception signal in which the clutter component is cut or suppressed is an example of the first filtered Doppler signal or the Doppler shift signal according to the present disclosure.

The wall motion filter for sound 3fs may be configured as, for example, a high-pass filter. The wall motion filter for sound 3fs performs, under the control of the filtering controller 12, filter processing of cutting or suppressing a low-frequency component (clutter component) included in the reception signal output from the accumulation circuit 3e on the basis of filter characteristics (for example, cutoff frequency or the like) applied for sound. Then, the wall motion filter for sound 3fs generates a reception signal in which the clutter component is cut or suppressed, and outputs the reception signal to the Doppler sound signal generator 3h. The reception signal in which the clutter component is cut or suppressed is an example of the second filtered Doppler signal or the Doppler shift signal according to the present disclosure.

Note that the wall motion filter for image 3fi and the wall motion filter for sound 3fs may be realized as separate filters as hardware or software modules, or may be realized as a single filter. In the latter case, the filtering controller 12 can realize the wall motion filter for image 3fi and the wall motion filter for sound 3fs by switching the filter characteristics of a single filter.

The FFT analyzer 3g generates a Doppler spectrum by performing frequency analysis on the Doppler shift frequency component of the reception signal output from the wall motion filter for image 3fi and outputs the Doppler spectrum to the display processor 4. The FFT analyzer 3g is an example of an analyzer according to the present disclosure. The Doppler spectrum is an example of an image signal, a spectrum signal, or a spectrum image signal according to the present disclosure.

The Doppler sound signal generator 3h separates the direction of the reception signal output from the wall motion filter for sound 3fs with respect to the ultrasonic probe 200, performs frequency conversion as necessary, and executes post-processing such as DA conversion, thereby generating a Doppler sound signal (for example, the stereo left signal and the stereo right signal) and outputting the signal to the Doppler sound output part 6. Note that the stereo left signal and the stereo right signal may be the same (that is, monophonic). The Doppler sound signal generator 3h is an example of a generator according to the present disclosure. The Doppler sound signal is an example of a sound signal according to the present disclosure.

[Filter Characteristics of Wall Motion Filter]

First, before describing filter characteristics of the wall motion filter according to the present embodiment, an example of applying a common wall motion filter to an image and sound will be described with reference to FIGS. 1B and 1C.

FIG. 1B is a diagram illustrating an example of a Doppler waveform, and illustrates an example in which a tissue component of a Doppler signal cannot be cut by a wall motion filter. In FIG. 1B, if there is a strong signal of tissue (a portion indicated by an arrow), the signal cannot be cut by the wall motion filter, and the Doppler sound is mixed with a popping noise.

In order to avoid this (that is, in order to cut low-frequency components), it is conceivable to increase the cutoff frequency of the wall motion filter in accordance with the Doppler sound.

FIG. 1C is a diagram illustrating an example of the Doppler waveform, and illustrates an example in which the cutoff frequency of the wall motion filter is increased. In a case where the cutoff frequency is increased in this manner, as illustrated in FIG. 1C, a gap is generated in the vicinity of the baseline of the image.

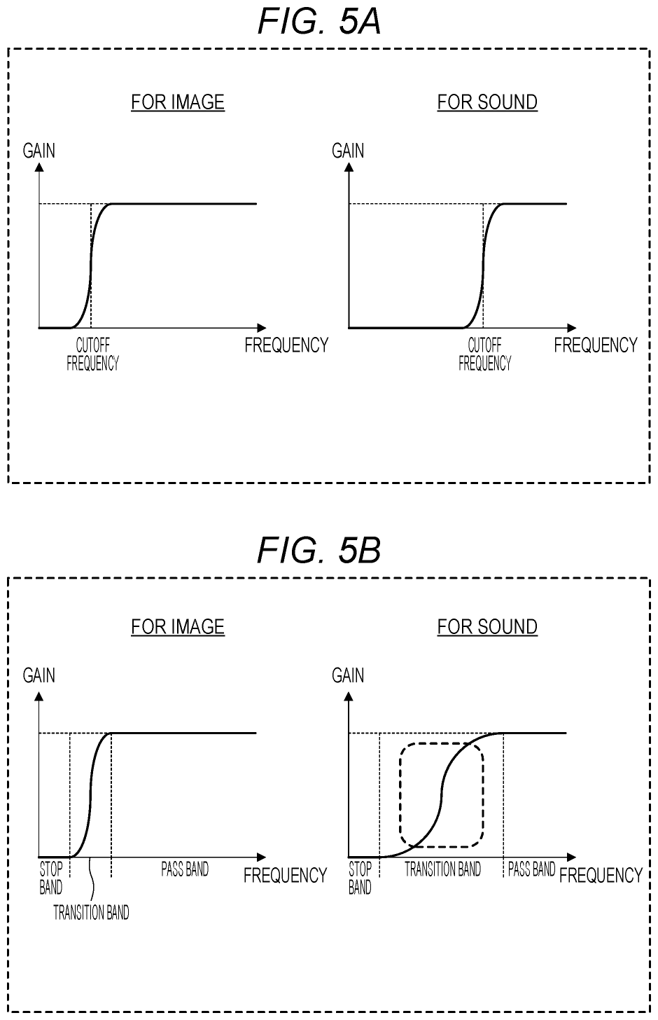

Hereinafter, an example of filter characteristics of the wall motion filter for image 3fi and the wall motion filter for sound 3fs for avoiding or suppressing such a problem will be described with reference to FIGS. 5A and 5B.

FIGS. 5A and 5B are diagrams illustrating examples of filter characteristics of the wall motion filter for image 3fi and the wall motion filter for sound 3fs.

As illustrated in FIG. 5A, the cutoff frequency (right side in FIG. 5A) of the wall motion filter for sound 3fs may be higher than the cutoff frequency (left side in FIG. 5A) of the wall motion filter for image 3fi. As a result, it is possible to prevent or suppress generation of a gap in the vicinity of the baseline of the image and prevent the Doppler sound from being mixed with the popping noise. Note that the cutoff frequency may be defined as, for example, a frequency or the like at which the cutoff characteristic of the filter is −6 dB.

Furthermore, as illustrated in FIG. 5B, the filter characteristics may be such that the gain change (right side in FIG. 5B) in the transition band of the wall motion filter for sound 3fs is gentler than the gain change (left side in FIG. 5B) in the transition band of the wall motion filter for image 3fi. As a result, it is possible to leave the low frequency sound while suppressing the popping noise, instead of cutting all the low frequency sound of the Doppler sound. For example, the low frequency sound in the portion surrounded by the dotted frame in FIG. 5B is not completely cut but left, so that the low frequency sound is expressed, and by lowering the gain, it is possible to suppress the popping noise.

Note that, in the case of the configuration illustrated in FIG. 5B, a user interface that allows the user to select the cutoff frequency of the wall motion filter for image 3fi may be provided. In addition, the values indicating the steepness of the transition band and the gain change of the transition band of the wall motion filter for sound 3fs associated with the respective cutoff frequencies of the wall motion filter for image 3fi may be determined in advance and stored in the ultrasonic diagnostic apparatus A.

[Cutoff Frequency Selection of Wall Motion Filter]

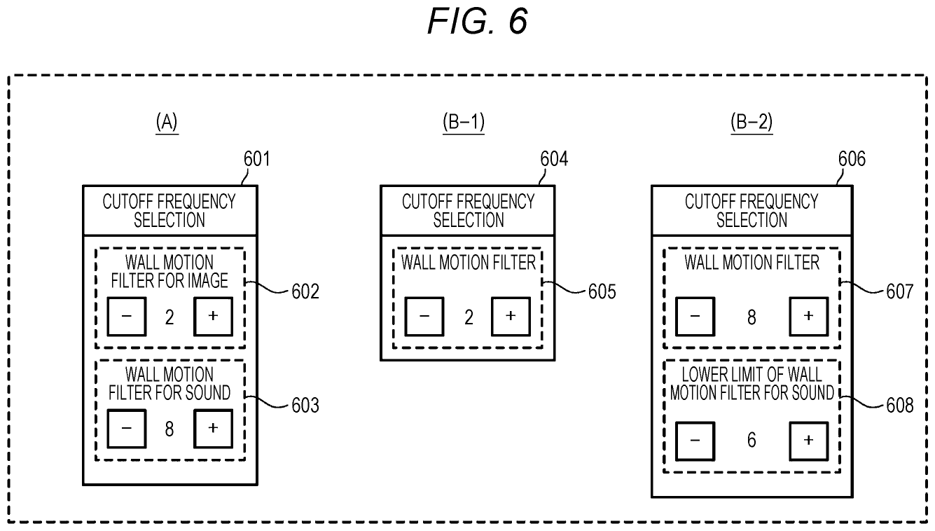

Hereinafter, an example of a user interface (screen) for cutoff frequency selection by the user will be described with reference to FIG. 6.

FIG. 6 illustrates an example user interface to allow the user to select the cutoff frequency (that is, the cutoff characteristic) of the wall motion filter. Such a user interface is displayed on the monitor 5 when the user operates a button, a keyboard, a mouse, or the like of the operation input part 8.

(A) of FIG. 6 illustrates a user interface 601 including a setting region 602 for setting the cutoff frequency of the wall motion filter for image 3fi and a setting region 603 for setting the cutoff frequency of the wall motion filter for sound 3fs. The setting region 602 is an example of a first setting part according to the present disclosure, and the setting region 603 is an example of a second setting part according to the present disclosure.

With respect to the cutoff frequency of the wall motion filter for image 3fi, for example, the user can increase (or decrease) the index value (the center in the setting region 602) associated with the cutoff frequency by positioning the mouse on the “+” button (or the “−” button) in the setting region 602 and clicking it. Furthermore, with respect to the cutoff frequency of the wall motion filter for sound 3fs, for example, the user can increase (or decrease) the index value (the center in the setting region 603) associated with the cutoff frequency by positioning the mouse on the “+” button (or the “−” button) in the setting region 603 and clicking it. Note that, for example, the user can reflect the setting by positioning the mouse on a button (not illustrated) for actually applying the cutoff frequency (for storing the button in the ultrasonic diagnostic apparatus A as setting information) and clicking the button. The same applies to the user interface described below.

The illustrated index value may be, for example, an integer of 1 to 10 for both the image and the sound, and a larger index value may be associated with a higher cutoff frequency. Information indicating the association between the index value and the cutoff frequency is stored in the ultrasonic diagnostic apparatus A. The same applies to the user interface described below.

In this example, the cutoff frequency associated with the index value “2” is applied to the cutoff frequency of the wall motion filter for image 3fi, and the cutoff frequency associated with the index value “8” is applied to the cutoff frequency of the wall motion filter for sound 3fs. Therefore, the cutoff frequency of the wall motion filter for sound 3fs is higher than the cutoff frequency of the wall motion filter for image 3fi.

(B-1) of FIG. 6 illustrates a user interface 604 including a setting region 605 for setting the cutoff frequency of the wall motion filter for image 3fi and the cutoff frequency of the wall motion filter for sound 3fs with a single (common) setting item. In this example, a lower limit value is provided for the cutoff frequency of the wall motion filter for sound 3fs, but this lower limit value is an internal parameter that cannot be set by the user. Note that the index value associated with the lower limit value of the cutoff frequency is assumed to be 6. The setting region 605 is an example of a third setting part according to the present disclosure.

For the cutoff frequency of the wall motion filter, for example, the user can increase (or decrease) the index value (the center in the setting region 605) associated with the cutoff frequency by positioning the mouse on the “+” button (or the “−” button) in the setting region 605 and clicking the button.

When the set cutoff frequency (index value) is equal to or more than the lower limit value, the set cutoff frequency is applied to both the cutoff frequency of the wall motion filter for image 3fi and the cutoff frequency of the wall motion filter for sound 3fs.

On the other hand, when the set cutoff frequency is lower than the lower limit value, the set cutoff frequency is applied to the cutoff frequency of the wall motion filter for image 3fi, but the lower limit value of the cutoff frequency is applied to the cutoff frequency of the wall motion filter for sound 3fs.

In this example, the cutoff frequency associated with the index value “2” is applied to the cutoff frequency of the wall motion filter for image 3fi. On the other hand, since the set index value “2” is smaller than the index value “6” associated with the lower limit value of the cutoff frequency, the cutoff frequency associated with the index value “6” is applied to the cutoff frequency of the wall motion filter for sound 3fs. Therefore, the cutoff frequency of the wall motion filter for sound 3fs is higher than the cutoff frequency of the wall motion filter for image 3fi.

Therefore, in this example, the cutoff frequency of the wall motion filter for sound 3fs is not lower than the cutoff frequency of the wall motion filter for image 3fi.

(B-2) of FIG. 6 illustrates a user interface 606 including a setting region 608 for setting the lower limit value of the cutoff frequency of the wall motion filter for sound 3fs in addition to a setting region 607 similar to the setting region 605 of (B-1) of FIG. 6. Thus, in this example, this lower limit value is settable by the user. The setting region 607 is an example of a third setting part according to the present disclosure, and the setting region 608 is an example of a fourth setting part according to the present disclosure.

As for the lower limit value of the cutoff frequency of the wall motion filter for sound 3fs, for example, the user can increase (or decrease) the index value associated with the lower limit value of the cutoff frequency by positioning the mouse on the “+” button (or the “−” button) in the setting region 608 and clicking it.

When the set cutoff frequency (index value) is equal to or more than the lower limit value (index value) of the set cutoff frequency, the set cutoff frequency is applied to both the cutoff frequency of the wall motion filter for image 3fi and the cutoff frequency of the wall motion filter for sound 3fs.

On the other hand, when the set cutoff frequency is lower than the lower limit value of the set cutoff frequency, the set cutoff frequency is applied to the cutoff frequency of the wall motion filter for image 3fi, but the lower limit value of the cutoff frequency is applied to the cutoff frequency of the wall motion filter for sound 3fs.

In this example, the cutoff frequency associated with the index value “8” is applied to the cutoff frequency of the wall motion filter for image 3fi. On the other hand, since the set index value “8” is larger than the index value “6” associated with the lower limit value of the cutoff frequency, the cutoff frequency associated with the index value “8” is also applied to the cutoff frequency of the wall motion filter for sound 3fs. Therefore, also in this example, the cutoff frequency of the wall motion filter for sound 3fs is not lower than the cutoff frequency of the wall motion filter for image 3fi.

[Operation of Ultrasonic Diagnostic Apparatus]

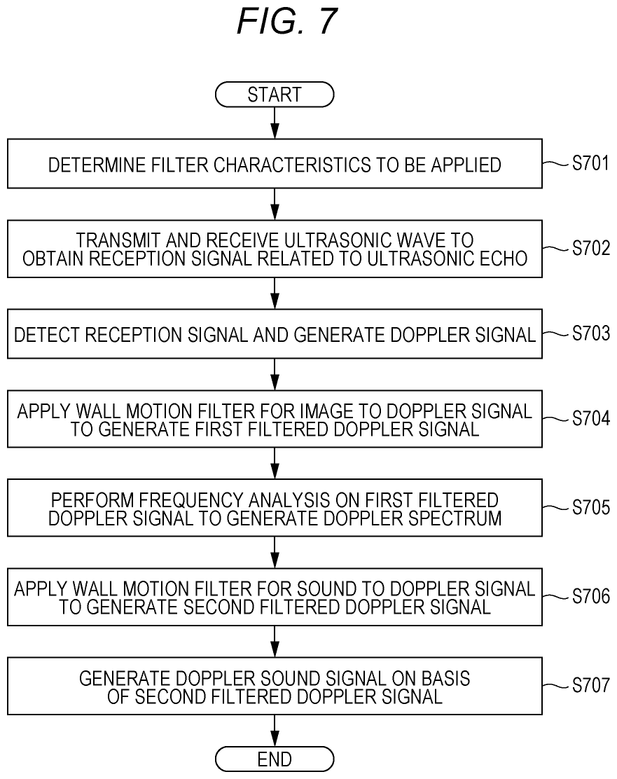

Hereinafter, an operation example of the ultrasonic diagnostic apparatus A will be described with reference to FIG. 7.

FIG. 7 is a flowchart illustrating an example of the operation of the ultrasonic diagnostic apparatus A according to the first embodiment.

In step S701, the wall motion filter 3f determines the filter characteristics of the wall motion filter for image 3fi and the wall motion filter for sound 3fs to be applied. As described above, the filter characteristics may be a cutoff frequency, steepness (moderation) of gain change in a transition band, or the like.

In step S702, the transceiver 1 transmits and receives an ultrasonic wave using the ultrasonic probe 200 to obtain a reception signal related to an ultrasonic echo from the observation target in the subject.

In step S703, the orthogonal detector 3b detects the reception signal and generates a Doppler signal.

In step S704, the wall motion filter 3f applies the wall motion filter for image 3fi having the filter characteristics determined in step S701 to the Doppler signal to generate the first filtered Doppler signal. That is, the wall motion filter for image 3fi generates the first filtered Doppler signal.

In step S705, the FFT analyzer 3g performs frequency analysis on the first filtered Doppler signal to generate a Doppler spectrum.

In step S706, the wall motion filter 3f applies the wall motion filter for sound 3fs having the filter characteristics determined in step S701 to the Doppler signal to generate the second filtered Doppler signal. That is, the wall motion filter for sound 3fs generates the second filtered Doppler signal.

In step S707, the Doppler sound signal generator 3h generates a Doppler sound signal on the basis of the second filtered Doppler signal.

Thereafter, the monitor 5 displays the display image generated by the display processor 4 on the basis of the Doppler spectrum, and the speaker 7 outputs the Doppler sound generated by the Doppler sound output part 6 on the basis of the Doppler sound signal.

Note that the steps in the flowchart illustrated in FIG. 7 are not limited to the illustrated order. For example, steps S704 and S706 may be executed in the reverse order or may be executed in parallel.

### Effects

As described above, the ultrasonic diagnostic apparatus (ultrasonic diagnostic apparatus A) according to the present embodiment includes:

a transceiver (transceiver 1) that transmits and receives an ultrasonic wave to obtain a reception signal related to an ultrasonic echo;

a detector (Doppler signal processor 3 and orthogonal detector 3b) that detects the reception signal and generates a Doppler signal;

a filtering part (wall motion filter 3f, wall motion filter for image 3fi, and wall motion filter for sound 3fs) that performs filter processing of cutting or suppressing a low-frequency component included in the Doppler signal and generates a first filtered Doppler signal and a second filtered Doppler signal from the Doppler signal;

an analyzer (FFT analyzer 3g) that performs frequency analysis on the first filtered Doppler signal to generate an image signal (Doppler spectrum); and

a generator (Doppler sound signal generator 3h) that generates a sound signal (Doppler sound signal) based on the second filtered Doppler signal.

According to the ultrasonic diagnostic apparatus (ultrasonic diagnostic apparatus A) according to the present embodiment, since the generator (Doppler sound signal generator 3h) can generate the sound signal (Doppler sound signal) without using the image signal (Doppler spectrum) generated by the analyzer (FFT analyzer 3g) performing the frequency analysis, it is possible to generate the Doppler sound from which the low-frequency clutter has been removed without complicating the processing.

### Second Embodiment

Next, a second embodiment of the present disclosure will be described. Hereinafter, in the second embodiment, portions different from those of the first embodiment will be described.

In the present embodiment, the Doppler signal processor 3 applies a wall motion filter for shared to the Doppler signal before generating the Doppler spectrum, and applies the wall motion filter for sound to the filtered Doppler signal obtained by applying the wall motion filter for shared to the Doppler signal before generating the Doppler sound signal. By applying the filter in this manner, a Doppler sound from which a low-frequency clutter has been removed is generated while avoiding the complexity of processing of generating a Doppler sound by performing inverse Fourier transform on a filtered signal.

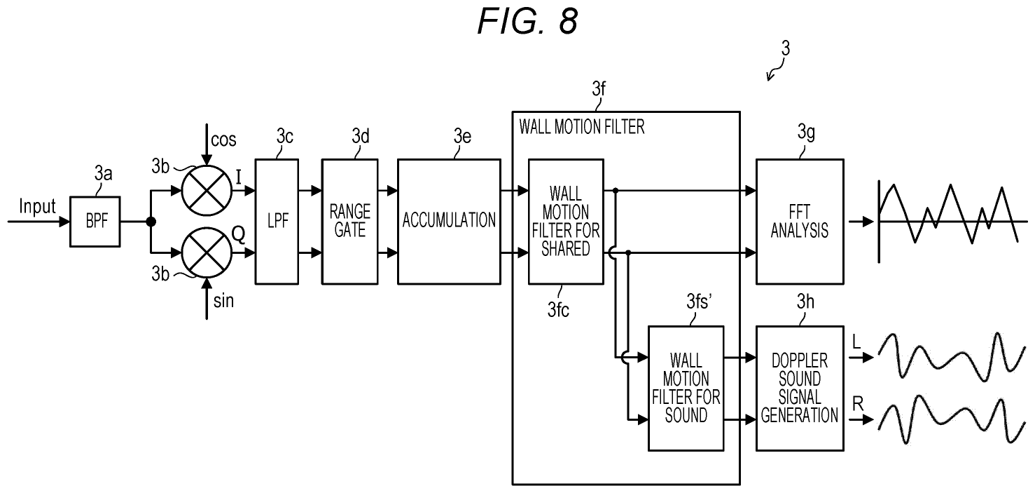

FIG. 8 is a diagram illustrating an example of a configuration of the Doppler signal processor 3 that executes PWD according to the second embodiment. The configuration of the wall motion filter 3f is different between the first embodiment and the second embodiment. Specifically, the wall motion filter 3f according to the second embodiment includes a wall motion filter for shared 3fc and a wall motion filter for sound 3fs′. Therefore, in the second embodiment, the wall motion filter 3f performs filter processing of applying the wall motion filter for shared 3fc and the wall motion filter for sound 3fs′ to cut or suppress a low-frequency component (clutter component) included in the reception signal output from the accumulation circuit 3e.

The wall motion filter for shared 3fc may be configured as, for example, a high-pass filter. The wall motion filter for shared 3fc performs, under the control of the filtering controller 12, filter processing for cutting or suppressing a low-frequency component (clutter component) included in the reception signal output from the accumulation circuit 3e on the basis of filter characteristics (for example, cutoff frequency or the like) applied for shared (image). Then, the wall motion filter for shared 3fc generates a reception signal in which the clutter component is cut or suppressed, and outputs the reception signal to the FFT analyzer 3g and the wall motion filter for sound 3fs′. The reception signal in which the clutter component is cut or suppressed is an example of the first filtered Doppler signal or the Doppler shift signal according to the present disclosure.

The wall motion filter for sound 3fs′ may be configured as, for example, a high-pass filter. The wall motion filter for sound 3fs′ performs, under the control of the filtering controller 12, filter processing of cutting or suppressing a low-frequency component (clutter component) included in the reception signal output from the wall motion filter for shared 3fc on the basis of filter characteristics (for example, cutoff frequency or the like) applied for sound. Then, the wall motion filter for sound 3fs′ generates a reception signal in which the clutter component is cut or suppressed, and outputs the reception signal to the Doppler sound signal generator 3h. The reception signal in which the clutter component is cut or suppressed is an example of the second filtered Doppler signal or the Doppler shift signal according to the present disclosure.

Note that the wall motion filter for shared 3fc and the wall motion filter for sound 3fs′ may be realized as separate filters as hardware or software modules, or may be realized as a single filter. In the latter case, the filtering controller 12 can realize the wall motion filter for shared 3fc and the wall motion filter for sound 3fs′ by switching the filter characteristics of a single filter.

[Filter Characteristics of Wall Motion Filter]

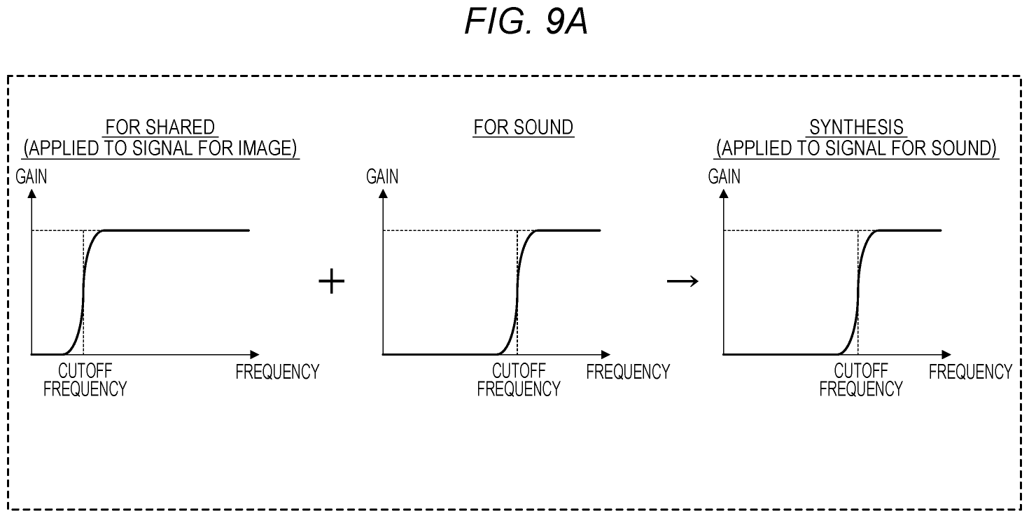

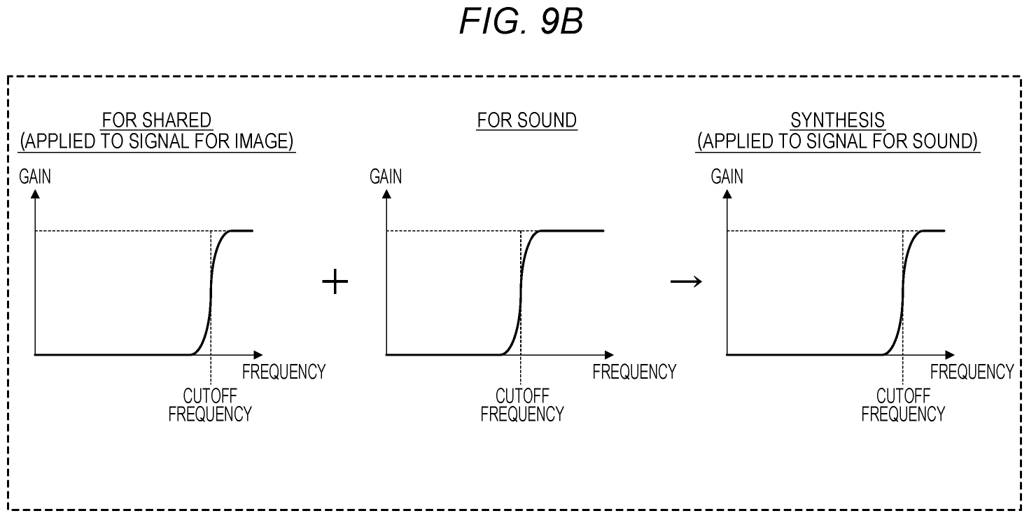

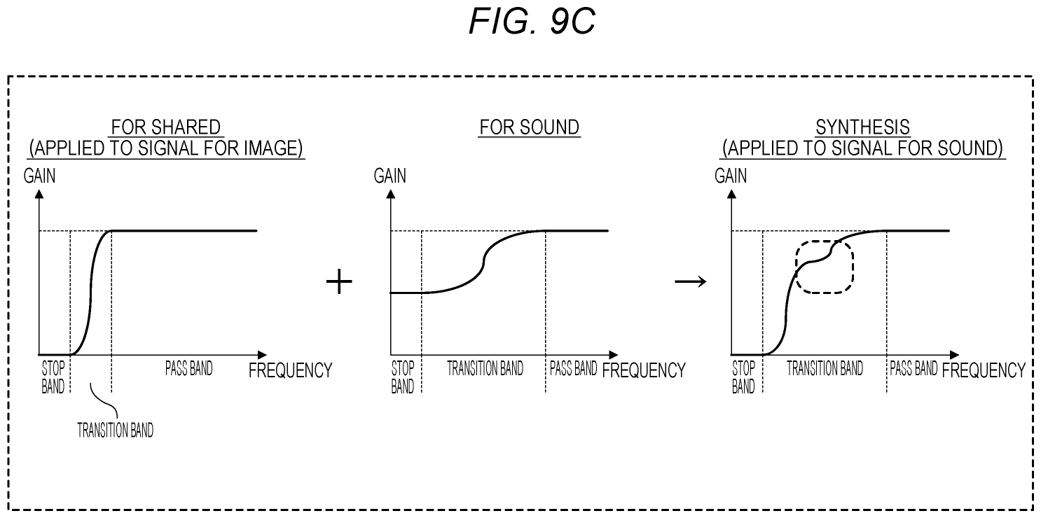

Hereinafter, an example of filter characteristics of the wall motion filter for shared 3fc and the wall motion filter for sound 3fs′ and filter characteristics obtained by synthesizing these filters will be described with reference to FIGS. 9A to 9C.

FIGS. 9A to 9C are diagrams illustrating examples of filter characteristics of the wall motion filter for shared 3fc and the wall motion filter for sound 3fs′ and filter characteristics obtained by synthesizing these filters.

As illustrated in FIG. 9A, the cutoff frequency (the center of FIG. 9A) of the wall motion filter for sound 3fs′ may be higher than the cutoff frequency (the left side of FIG. 9A) of the wall motion filter for shared 3fc. As a result, the cutoff frequency of the wall motion filter for sound 3fs′ is applied for sound (right side in FIG. 9A). Therefore, it is possible to prevent the popping noise from being mixed with the Doppler sound while preventing or suppressing the generation of the gap near the baseline of the image. Note that the cutoff frequency may be defined as, for example, a frequency or the like at which the cutoff characteristic of the filter is −6 dB.

Furthermore, if the cutoff frequency of the wall motion filter for shared 3fc is changed while the cutoff frequency of the wall motion filter for sound 3fs′ is fixed, an operation similar to the case of providing the lower limit value for the cutoff frequency of the wall motion filter for sound 3fs described above can be performed. That is, the cutoff frequency of the wall motion filter for sound 3fs′ can function as the lower limit value of the cutoff frequency of the wall motion filter for sound 3fs described above. For example, as illustrated in FIG. 9B, in a case where the cutoff frequency of the wall motion filter for shared 3fc (the left side of FIG. 9B) is higher than the cutoff frequency of the wall motion filter for sound 3fs′ (the center of FIG. 9B), the cutoff frequency of the wall motion filter for shared 3fc is applied for sound (the right side of FIG. 9B).

In addition, filter characteristics may be such that the gain change in the transition band of the wall motion filter for sound 3fs′ is gentler than the gain change in the transition band of the wall motion filter for shared 3fc. As a result, it is possible to leave the low frequency sound while suppressing the popping noise, instead of cutting all the low frequency sound of the Doppler sound. Alternatively or additionally, filter characteristics may be such that the gain of the stop band of the wall motion filter for sound 3fs′ is higher than the gain of the stop band of the wall motion filter for shared 3fc. As a result, it is possible to balance between suppressing the popping noise and leaving the low frequency sound by the gain of the stop band of the wall motion filter for sound 3fs′. FIG. 9C illustrates an example in which both of these filter characteristics are applied. As illustrated in FIG. 9C, when the gain change in the transition band of the wall motion filter for sound 3fs′ is gentler than the gain change in the transition band of the wall motion filter for shared 3fc, and the gain in the stop band of the wall motion filter for sound 3fs′ is higher than the gain in the stop band of the wall motion filter for shared 3fc (the left side and the center in FIG. 9C), for example, the low frequency sound of the portion surrounded by the dotted frame (the right side in FIG. 9C) can be left without being completely cut.

Note that, in the case of the configuration illustrated in FIG. 9C, a user interface that allows the user to select the cutoff frequency of the wall motion filter for shared 3fc and the gain of the stop band of the wall motion filter for sound 3fs′ may be provided. In addition, values indicating the transition band and steepness of the gain change in the transition band of the wall motion filter for sound 3fs associated with each combination of the cutoff frequency of the wall motion filter for image 3fi and the gain of the stop band of the wall motion filter for sound 3fs′ may be determined in advance and stored in the ultrasonic diagnostic apparatus A.

[Cutoff Frequency Selection of Wall Motion Filter]

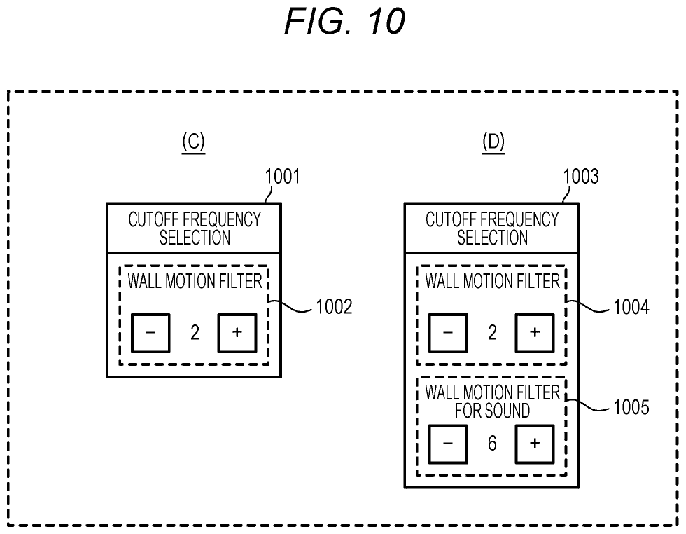

Hereinafter, an example of a user interface (screen) for cutoff frequency selection by the user will be described with reference to FIG. 10.

FIG. 10 illustrates an example user interface to allow the user to select the cutoff frequency (that is, the cutoff characteristic) of the wall motion filter. Such a user interface is displayed on the monitor 5 when the user operates a button, a keyboard, a mouse, or the like of the operation input part 8.

(C) of FIG. 10 illustrates a user interface 1001 including a setting region 1002 for setting the cutoff frequency of the wall motion filter for shared 3fc. In this example, the cutoff frequency of the wall motion filter for sound 3fs′ is an internal parameter that cannot be set by the user. The setting region 1002 is an example of a fifth setting part according to the present disclosure. Note that the operation and the like on the user interface 1001 illustrated in (C) of FIG. 10 are similar to the operation and the like on the user interface 604 illustrated in (B-1) of FIG. 6, and thus, description thereof is omitted.

(D) of FIG. 10 illustrates a user interface 1003 including a setting region 1005 for setting the cutoff frequency of the wall motion filter for sound 3fs′ in addition to a setting region 1004 similar to the setting region 1002 in (C) of FIG. 10. In this example, the cutoff frequency of the wall motion filter for sound 3fs′ can be set by the user. The setting region 1004 is an example of a fifth setting part according to the present disclosure, and the setting region 1005 is an example of a sixth setting part according to the present disclosure. Note that the operation and the like on the user interface 1003 illustrated in (D) of FIG. 10 are similar to the operation and the like on the user interface 601 illustrated in (A) of FIG. 6, and thus, description thereof is omitted.

[Operation of Ultrasonic Diagnostic Apparatus]

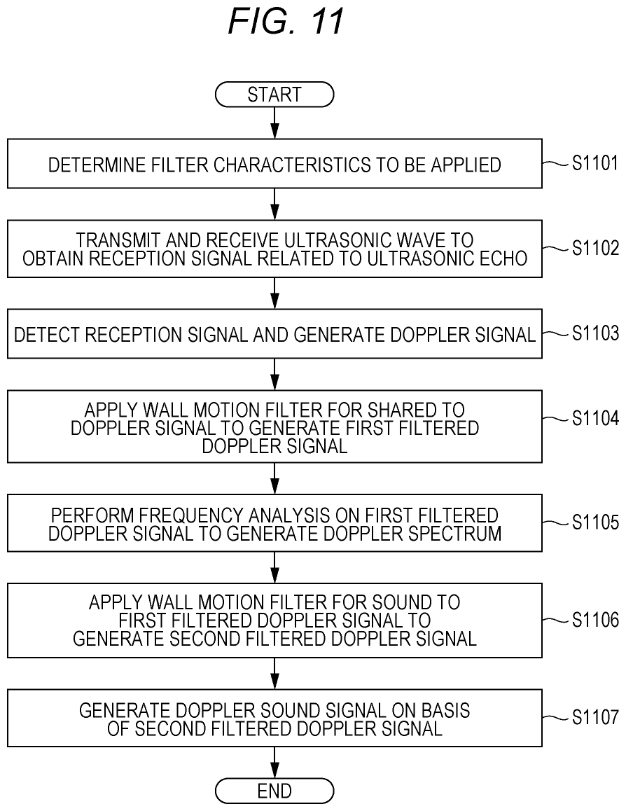

Hereinafter, an operation example of the ultrasonic diagnostic apparatus A will be described with reference to FIG. 11.

FIG. 11 is a flowchart illustrating an example of the operation of the ultrasonic diagnostic apparatus A according to the second embodiment.

In step S1101, the wall motion filter 3f determines the filter characteristics of the wall motion filter for shared 3fc and the wall motion filter for sound 3fs′ to be applied. As described above, the filter characteristics may be a cutoff frequency, steepness (moderation) of gain change in a transition band, or the like.

In step S1102, the transceiver 1 transmits and receives an ultrasonic wave using the ultrasonic probe 200 to obtain a reception signal related to an ultrasonic echo from the observation target in the subject.

In step S1103, the orthogonal detector 3b detects the reception signal and generates a Doppler signal.

In step S1104, the wall motion filter 3f applies the wall motion filter for shared 3fc having the filter characteristics determined in step S1101 to the Doppler signal to generate the first filtered Doppler signal That is, the wall motion filter for shared 3fc generates the first filtered Doppler signal.

In step S1105, the FFT analyzer 3g performs frequency analysis on the first filtered Doppler signal to generate a Doppler spectrum.

In step S1106, the wall motion filter 3f applies the wall motion filter for sound 3fs′ having the filter characteristics determined in step S1101 to the first filtered Doppler signal to generate the second filtered Doppler signal. That is, the wall motion filter for sound 3fs′ generates the second filtered Doppler signal.

In step S1107, the Doppler sound signal generator 3h generates a Doppler sound signal on the basis of the second filtered Doppler signal.

Thereafter, the monitor 5 displays the display image generated by the display processor 4 on the basis of the Doppler spectrum, and the speaker 7 outputs the Doppler sound generated by the Doppler sound output part 6 on the basis of the Doppler sound signal.

### Effects

As described above, the ultrasonic diagnostic apparatus (ultrasonic diagnostic apparatus A) according to the present embodiment includes:

a transceiver (transceiver 1) that transmits and receives an ultrasonic wave to obtain a reception signal related to an ultrasonic echo;

a detector (Doppler signal processor 3 and orthogonal detector 3b) that detects the reception signal and generates a Doppler signal;

a filtering part (wall motion filter 3f, wall motion filter for shared 3fc, and wall motion filter for sound 3fs′) that performs filter processing of cutting or suppressing a low-frequency component included in the Doppler signal and generates a first filtered Doppler signal and a second filtered Doppler signal from the Doppler signal;

an analyzer (FFT analyzer 3g) that performs frequency analysis on the first filtered Doppler signal to generate an image signal; and

a generator (Doppler sound signal generator 3h) that generates a sound signal based on the second filtered Doppler signal.

According to the ultrasonic diagnostic apparatus (ultrasonic diagnostic apparatus A) according to the present embodiment, since the generator (Doppler sound signal generator 3h) can generate the sound signal (Doppler sound signal) without using the image signal (Doppler spectrum) generated by the analyzer (FFT analyzer 3g) performing the frequency analysis, it is possible to generate the Doppler sound from which the low-frequency clutter has been removed without complicating the processing.

(First Modification)

In the user interface 601 described above, the user can also set the cutoff frequency of the wall motion filter for image 3fi to be higher than the cutoff frequency of the wall motion filter for sound 3fs. Also in the user interface 1001, the user can set the cutoff frequency of the wall motion filter for shared 3fc to be higher than the cutoff frequency of the wall motion filter for sound 3fs′. However, as in other examples of the user interface, the cutoff frequency of the wall motion filter for sound 3fs (or the wall motion filter for sound 3fs′) may not be lower than the cutoff frequency of the wall motion filter for image 3fi (or the wall motion filter for shared 3fc). For example, in a case where the cutoff frequency (index value) of the wall motion filter for image 3fi is higher (larger) than the cutoff frequency (index value) of the wall motion filter for sound 3fs, when the user clicks a button for actually applying the cutoff frequency, the ultrasonic diagnostic apparatus A may display a pop-up message indicating that the setting cannot be reflected on the monitor 5.

(Second Modification)

In the user interface 601 and the like described above, the cutoff frequency is selected by the user selecting the index value, but instead of the index value, a plurality of cutoff frequencies may be displayed in the form of options, and the user may select one of the plurality of options. Alternatively, instead of the index value, the user may input the cutoff frequency itself.

(Third Modification)

In the example illustrated in FIG. 5B, the values indicating the transition band and the steepness of the gain change in the transition band of the wall motion filter for sound 3fs are predetermined and stored in the ultrasonic diagnostic apparatus A. However, the values indicating the transition band and the steepness of the gain change in the transition band may be displayed in the form of options, and the user may select one of the plurality of options. Alternatively, the user may be able to input the values themselves indicating the transition band and the steepness of the gain change in the transition band.

### Summary of Embodiments

An ultrasonic diagnostic apparatus according to an aspect of the present disclosure includes: a transceiver that transmits and receives an ultrasonic wave to obtain a reception signal related to an ultrasonic echo; a detector that detects the reception signal and generates a Doppler signal; a filtering part that performs filter processing of cutting or suppressing a low-frequency component included in the Doppler signal and generates a first filtered Doppler signal and a second filtered Doppler signal from the Doppler signal; an analyzer that performs frequency analysis on the first filtered Doppler signal to generate an image signal; and a generator that generates a sound signal based on the second filtered Doppler signal.

With the above configuration, since a sound signal can be generated without using an image signal

generated by frequency analysis, it is possible to generate a Doppler sound from which a low-frequency clutter has been removed without complicating processing.

In the ultrasonic diagnostic apparatus, the filtering part applies a first filter to the Doppler signal to generate the first filtered Doppler signal, and applies a second filter having a filter characteristic different from a filter characteristic of the first filter to the Doppler signal to generate the second filtered Doppler signal.

With the above configuration, it is possible to apply a filter having a filter characteristic corresponding to each of the spectrum image and the Doppler sound.

In the ultrasonic diagnostic apparatus, the first filter and the second filter are configured as high-pass filters, and a cutoff frequency of the second filter is higher than a cutoff frequency of the first filter.

With the above configuration, it is possible to cut the low-frequency component with respect to the Doppler sound and not to cut the signal much in the vicinity of the baseline with respect to the spectral image so as not to mix the popping noise.

In the ultrasonic diagnostic apparatus, the first filter and the second filter are configured as high-pass filters, and a gain change in a transition band of the second filter is gentler than a gain change in a transition band of the first filter.

With the above configuration, it is possible to suppress the popping noise by lowering the gain of the low frequency sound while expressing the low sound by leaving the low frequency sound without completely cutting the low sound.

The ultrasonic diagnostic apparatus further includes: a first setting part operable by a user to set the filter characteristic of the first filter; and a second setting part operable by the user to set the filter characteristic of the second filter.

With the above configuration, the user can adjust the filter characteristic according to the spectral image and the Doppler sound.

In the ultrasonic diagnostic apparatus, the filter characteristic of the first filter and the filter characteristic of the second filter are cutoff frequencies.

With the above configuration, the user can adjust the cutoff frequency according to each of the spectrum image and the Doppler sound.

The ultrasonic diagnostic apparatus further includes a third setting part operable by a user to set cutoff frequencies of the first filter and the second filter, in which a lower limit value is provided for the cutoff frequency of the second filter, and when the cutoff frequency set of the second filter is lower than the lower limit value, the filtering part applies the second filter having the cutoff frequency of the lower limit value, and generates the second filtered Doppler signal.

With the above configuration, the user can apply the cutoff frequencies of the two filters with a simple operation of setting one cutoff frequency.

The ultrasonic diagnostic apparatus further includes a fourth setting part operable by the user to set the lower limit value.

With the above configuration, the user can set a low frequency band to be cut in the Doppler sound.

In the ultrasonic diagnostic apparatus, the filtering part applies a third filter to the Doppler signal to generate the first filtered Doppler signal, and applies a fourth filter having a filter characteristic different from a filter characteristic of the third filter to the first filtered Doppler signal to generate the second filtered Doppler signal.

With the above configuration, it is possible to apply a filter having a filter characteristic corresponding to each of the spectrum image and the Doppler sound.

In the ultrasonic diagnostic apparatus, the third filter and the fourth filter are configured as high-pass filters, and a cutoff frequency of the fourth filter is higher than a cutoff frequency of the third filter.

With the above configuration, it is possible to cut the low-frequency component with respect to the Doppler sound and not to cut the signal much in the vicinity of the baseline with respect to the spectral image so as not to mix the popping noise.

In the ultrasonic diagnostic apparatus, the third filter and the fourth filter are configured as high-pass filters, and a gain change in a transition band of the fourth filter is gentler than a gain change in a transition band of the third filter.

With the above configuration, it is possible to suppress the popping noise by lowering the gain of the low frequency sound while expressing the low sound by leaving the low frequency sound without completely cutting the low sound.

In the ultrasonic diagnostic apparatus, the third filter and the fourth filter are configured as high-pass filters, and a gain of a stop band of the fourth filter is higher than a gain of a stop band of the third filter.

With the above configuration, it is possible to balance between suppressing the popping noise and leaving the low frequency sound by the gain of the stop band of the fourth filter.

The ultrasonic diagnostic apparatus further includes: a fifth setting part operable by the user to set the filter characteristic of the third filter; and a sixth setting part operable by the user to set the filter characteristic of the fourth filter.

With the above configuration, the user can adjust the filter characteristic according to the spectral image and the Doppler sound.

In the ultrasonic diagnostic apparatus, the filter characteristic of the third filter and the filter characteristic of the fourth filter are cutoff frequencies.

With the above configuration, the user can adjust the cutoff frequency according to each of the spectrum image and the Doppler sound.

A method for controlling an ultrasonic diagnostic apparatus according to an aspect of the present disclosure includes: transmitting and receiving an ultrasonic wave to obtain a reception signal related to an ultrasonic echo; detecting the reception signal to generate a Doppler signal; performing filter processing of cutting or suppressing a low-frequency component included in the Doppler signal and generating a first filtered Doppler signal and a second filtered Doppler signal from the Doppler signal; performing frequency analysis on the first filtered Doppler signal to generate an image signal; and generating a sound signal based on the second filtered Doppler signal.

With the above configuration, since a sound signal can be generated without using an image signal generated by frequency analysis, it is possible to generate a Doppler sound from which a low-frequency clutter has been removed without complicating processing.

A non-transitory recording medium storing a computer readable control program of an ultrasonic diagnostic apparatus according to an aspect of the present disclosure causes the ultrasonic diagnostic apparatus to execute: transmitting and receiving an ultrasonic wave to obtain a reception signal related to an ultrasonic echo; detecting the reception signal to generate a Doppler signal; performing filter processing of cutting or suppressing a low-frequency component included in the Doppler signal and generating a first filtered Doppler signal and a second filtered Doppler signal from the Doppler signal; performing frequency analysis on the first filtered Doppler signal to generate an image signal; and generating a sound signal based on the second filtered Doppler signal.

With the above configuration, since a sound signal can be generated without using an image signal generated by frequency analysis, it is possible to generate a Doppler sound from which a low-frequency clutter has been removed without complicating processing.

An aspect of the present disclosure is useful as an ultrasonic diagnostic apparatus capable of generating a Doppler sound from which a low-frequency clutter has been removed without complicating processing.

Although embodiments of the present invention have been described and illustrated in detail, the disclosed embodiments are made for purposes of illustration and example only and not limitation. The scope of the present invention should be interpreted by terms of the appended claims. The technology described in the claims includes various modifications and changes of the specific examples exemplified above.

What is claimed is:

1. An ultrasonic diagnostic apparatus comprising:
a transceiver that transmits and receives an ultrasonic wave to obtain a reception signal related to an ultrasonic echo;
a detector that detects the reception signal and generates a Doppler signal;
a filtering part that performs filter processing of cutting or suppressing a low-frequency component included in the Doppler signal and generates a first filtered Doppler signal and a second filtered Doppler signal from the Doppler signal;
an analyzer that performs frequency analysis on the first filtered Doppler signal to generate an image signal; and
a generator that generates a sound signal based on the second filtered Doppler signal.

2. The ultrasonic diagnostic apparatus according to claim 1, wherein
the filtering part
applies a first filter to the Doppler signal to generate the first filtered Doppler signal, and
applies a second filter having a filter characteristic different from a filter characteristic of the first filter to the Doppler signal to generate the second filtered Doppler signal.

3. The ultrasonic diagnostic apparatus according to claim 2, wherein
the first filter and the second filter include high-pass filters, and
a cutoff frequency of the second filter is higher than a cutoff frequency of the first filter.

4. The ultrasonic diagnostic apparatus according to claim 2, wherein
the first filter and the second filter include high-pass filters, and
a gain change in a transition band of the second filter is gentler than a gain change in a transition band of the first filter.

5. The ultrasonic diagnostic apparatus according to claim 2, further comprising:
a first setting part operable by a user to set the filter characteristic of the first filter; and
a second setting part operable by the user to set the filter characteristic of the second filter.

6. The ultrasonic diagnostic apparatus according to claim 5, wherein
the filter characteristic of the first filter and the filter characteristic of the second filter are cutoff frequencies.

7. The ultrasonic diagnostic apparatus according to claim 2, further comprising
a third setting part operable by a user to set cutoff frequencies of the first filter and the second filter, wherein
a lower limit value is provided for the cutoff frequency of the second filter, and
when the set cutoff frequency of the second filter is lower than the lower limit value, the filtering part applies the second filter having the cutoff frequency of the lower limit value, and generates the second filtered Doppler signal.

8. The ultrasonic diagnostic apparatus according to claim 7, further comprising
a fourth setting part operable by the user to set the lower limit value.

9. The ultrasonic diagnostic apparatus according to claim 1, wherein
the filtering part
applies a third filter to the Doppler signal to generate the first filtered Doppler signal, and
applies a fourth filter having a filter characteristic different from a filter characteristic of the third filter to the first filtered Doppler signal to generate the second filtered Doppler signal.

10. The ultrasonic diagnostic apparatus according to claim 9, wherein
the third filter and the fourth filter include high-pass filters, and
a cutoff frequency of the fourth filter is higher than a cutoff frequency of the third filter.

11. The ultrasonic diagnostic apparatus according to claim 9, wherein
the third filter and the fourth filter include high-pass filters, and
a gain change in a transition band of the fourth filter is gentler than a gain change in a transition band of the third filter.

12. The ultrasonic diagnostic apparatus according to claim 9, wherein
the third filter and the fourth filter include high-pass filters, and
a gain of a stop band of the fourth filter is higher than a gain of a stop band of the third filter.

13. The ultrasonic diagnostic apparatus according to claim 9, further comprising:
a fifth setting part operable by the user to set the filter characteristic of the third filter; and
a sixth setting part operable by the user to set the filter characteristic of the fourth filter.

14. The ultrasonic diagnostic apparatus according to claim 13, wherein
the filter characteristic of the third filter and the filter characteristic of the fourth filter are cutoff frequencies.

15. A method for controlling an ultrasonic diagnostic apparatus, the method comprising:
transmitting and receiving an ultrasonic wave to obtain a reception signal related to an ultrasonic echo;
detecting the reception signal to generate a Doppler signal;
performing filter processing of cutting or suppressing a low-frequency component included in the Doppler signal and generating a first filtered Doppler signal and a second filtered Doppler signal from the Doppler signal;
performing frequency analysis on the first filtered Doppler signal to generate an image signal; and
generating a sound signal based on the second filtered Doppler signal.

16. A non-transitory recording medium storing a computer readable control program of an ultrasonic diagnostic apparatus, the control program causing the ultrasonic diagnostic apparatus to execute:
transmitting and receiving an ultrasonic wave to obtain a reception signal related to an ultrasonic echo;
detecting the reception signal to generate a Doppler signal;
performing filter processing of cutting or suppressing a low-frequency component included in the Doppler signal and generating a first filtered Doppler signal and a second filtered Doppler signal from the Doppler signal;
performing frequency analysis on the first filtered Doppler signal to generate an image signal; and
generating a sound signal based on the second filtered Doppler signal.
